(19) United States
(12) Patent Application Publication
(10) Pub. No.: US 2022/0409934 A1
(43) Pub. Date: Dec. 29, 2022

(54) INTRA-KIDNEY STONE DISRUPTOR

(71) Applicant: GYRUS ACMI, INC. D/B/A OLYMPUS SURGICAL TECHNOLOGIES AMERICA, Westborough, MA (US)

(72) Inventor: Peter J. Crowley, Norfolk, MA (US)

(21) Appl. No.: 17/808,325

(22) Filed: Jun. 23, 2022

Related U.S. Application Data
(60) Provisional application No. 63/214,602, filed on Jun. 24, 2021.

(51) Int. Cl. A61N 7/00; A61B 1/307

(52) U.S. Cl. CPC A61N 7/00; A61B 1/307; A61N 2007/0004

(57) ABSTRACT

A medical apparatus for mitigating formation of kidney stones in a human patient can include or use a turbulence generator deployable into a renal pelvis of a human kidney, the generator comprising an element configured to produce an acoustic wave in a medium within the renal pelvis, and an actuator configured such as to manipulate the turbulence generator; wherein one of the turbulence generator or the actuator can be configured for coupling with a source of power.

### CROSS-REFERENCE TO RELATED APPLICATION

This application claims the benefit of priority to U.S. Provisional Patent Application Ser. No. 63/214,602, filed Jun. 24, 2021, the contents of which are hereby incorporated by reference in their entirety.

### TECHNICAL FIELD

This document pertains generally, but not by way of limitation, to apparatus and methods for mitigation of kidney stone formation.

### BACKGROUND

Kidney stones can develop in a human kidney and can migrate to the urinary tract. While small kidney stones can pass through the ureter without causing issues, stones greater than 5 millimeters in diameter can cause blockage of the ureter and can result in severe pain. A stone may also result in blood in the urine, vomiting, or painful urination. Stones can contain calcium oxalate, either alone or in combination with calcium phosphate in the form of apatite or brushite, struvite (ammonium magnesium phosphate), uric acid, cystine, xanthine, glycine, proline, hydroxyproline, or other bioelements. A medical procedure can be used to help mitigate stone formation or to breakup stones that have partially formed such as to help reduce the need for painful passing of stones or for other medical procedures to facilitate removal of the stones. Several specific therapies can be used to chemically mitigate or disrupt formation of kidney stones based on the chemical composition thereof.

### SUMMARY

In an approach to mitigation or disruption of kidney stones, a soft and atraumatic mechanism can be deployed in a human kidney. The mechanism can be introduced into the kidney by injection, such as injection including or using a ureteroscope. The mechanism can create motion such as to cause turbulence of fluid within the kidney. Such turbulence can reduce formation of kidney stones or can dislodge or fragment the stones. In some examples, the mechanism can oscillate, rotate, pulsate, spin, or vibrate such as to help create turbulence.

Aspect 1 can include or use a medical apparatus for mitigating kidney stones in a human patient, and the apparatus can include or use a turbulence generator deployable into a renal pelvis of a human kidney, the generator including or using an element configured such as to produce an acoustic wave in a medium within the renal pelvis, and an actuator configured such as to manipulate the turbulence generator. In Aspect 2, the medical apparatus of Aspect 1 can optionally be configured such that one of the turbulence generator or the actuator can be configured for coupling with a source of power. In Aspect 3, the medical apparatus of Aspect 1 and/or Aspect 2 can be optionally configured such that the element can include or use a chassis, an electric motor, and an eccentric weight disposed on a drive shaft of the electric motor, wherein rotation of the eccentric weight by the drive shaft can cause oscillation. In In Aspect 4, the medical apparatus of any one or any combination of Aspects 1-3 can be optionally configured such that the element can include or use a chassis, a linear actuator, and a weight configured such as to be oscillated along a linear path by the linear actuator. In Aspect 5, the medical apparatus of any one or any combination of Aspects 1-4 can optionally be configured such that the element is an acoustic transducer. In Aspect 6, the medical apparatus of any one or any combination of Aspects 1-5 can optionally be configured such that the element comprises electromagnetic coils. In Aspect 7, the medical apparatus of any one or any combination of Aspects 1-6 can optionally be configured such that the element is a piezoelectric vibrator. In Aspect 8, the medical apparatus of any one or any combination of Aspects 1-7 can optionally be configured such that the actuator is located within an external manipulator, and the manipulator can include or use a housing, a plurality of magnetic drives located within the housing, and a controller that selectively actuates the magnetic drives in accordance with an actuation sequence, wherein the element can be configured such as to move in response to the magnetic drives being actuated such as to stir the medium within the renal pelvis. In Aspect 9, the medical apparatus of any one or any combination of Aspects 1-8 can optionally be configured such that the manipulator can be configured such as to be located outside of a human body to actuate the element inside the renal pelvis of the human kidney. In Aspect 10, the medical apparatus of any one or any combination of Aspects 1-9 can optionally be configured such that the manipulator can be wearable by a human patient during actuation. In Aspect 11, the medical apparatus of any one or any combination of Aspects 1-10 can optionally be configured such that the manipulator comprises a belt configured such as for fastening to a human patient. In Aspect 12, the medical apparatus of any one or any combination of Aspects 1-11 can optionally be configured such that the element is a magnetic stir element configured to move in response to the magnetic drives of the housing. In Aspect 13, the medical apparatus of any one or any combination of Aspects 1-12 can optionally be configured such that the element can be coin-shaped. In Aspect 14, the medical apparatus of any one or any combination of Aspects 1-13 can optionally be configured such that the element is pill-shaped. In Aspect 15, the medical apparatus of any one or any combination of Aspects 1-14 can optionally be configured such that the element comprises a non-symmetrical shape. In Aspect 16, the medical apparatus of any one or any combination of Aspects 1-15 can optionally include or use a binding configured such as to anchor the turbulence generator to a predetermined location within the kidney. In Aspect 17, the medical apparatus of any one or any combination of Aspects 1-16 can optionally be configured such that the element can produce acoustic waves at frequencies between about 150 Hz and about 350 Hz. In Aspect 18, the medical apparatus of any one or any combination of Aspects 1-17 can optionally be configured such that the element produces ultrasonic waves at frequencies greater than about 20,000 Hz. In Aspect 19 can include or use a medical apparatus for mitigating kidney stones in a human patient, and the apparatus can include or use a turbulence generating element implantable into a renal pelvis of a human kidney and configured such as to passively generate turbulence in a medium within the renal pelvis, wherein the element can be suspended within the medium and at least one of gravitational or contact forces propel the element to generate the turbulence. In Aspect 20, the medical apparatus of Aspect 19 can optionally include or use a binding configured to anchor the turbulence generating element to a predetermined location within the kidney. In Aspect 21, the medical apparatus of any one or any combination of Aspects 1-20 can optionally include or use a method for treating a human patient, and the method can include or use deploying a turbulence generator into a renal pelvis of a kidney of the patient, activating the turbulence generator, generating mechanical turbulence in a medium within a renal pelvis, wherein the medium comprises a naturally-occurring fluid within the renal pelvis before the medium is activated, and circulating the medium out of calyxes into a main body of the renal pelvis and down a ureter. In Aspect 22, the medical apparatus of any one or any combination of Aspects 1-21 can optionally be configured such that generating mechanical turbulence can include or use administering a Lorentz force or an electromagnetic force from the turbulence generator and to the medium within the renal pelvis. In Aspect 23, the medical apparatus of any one or any combination of Aspects 1-22 can optionally be configured such that the turbulence generator is deployed into the renal pelvis using a ureteroscope. Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals can describe similar components in different views. Like numerals having different letter suffixes may represent different instances of similar components. The drawings illustrate generally, by way of example, but not by way of limitation, various embodiments discussed in the present document.

FIG. 1 is a side view of a stone disruptor in operation.

FIG. 2A is a perspective view of a portion of a stone disruptor.

FIG. 2B is a side view of a portion of a stone disruptor.

FIG. 3A is a perspective view of a portion of a stone disruptor.

FIG. 3B is a side view of a portion of a stone disruptor.

FIG. 4A is a perspective view of a portion of a stone disruptor.

FIG. 4B is a side view of a portion of a stone disruptor.

FIG. 5A is a perspective view of a portion of a stone disruptor.

FIG. 5B is a side view of a stone disruptor in operation.

FIG. 6A is a side view of a stone disruptor in operation.

FIG. 6B is a perspective view of a stone disruptor.

FIG. 6C is a side view of a stone disruptor.

### DETAILED DESCRIPTION

The present disclosure, in one or more examples, relates to apparatus and methods for mitigation of stone formation. More particularly, the present disclosure relates to an apparatus and method of use for creating turbulence within a human kidney. Kidney stones are generally formed when certain minerals exist in the urine at a high concentration. In one example, the urine can become supersaturated with one or more crystal forming substances, and a crystal can form through nucleation. Other biological or chemical processes can occur such as to cause stone formation, and the stones can be at least partially formed of calcium oxalate, either alone or in combination with calcium phosphate in the form of apatite or brushite, struvite (ammonium magnesium phosphate), uric acid, cystine, xanthine, glycine, proline, hydroxyproline, or other bioelements. Once a stone has become at least partially formed, the stone can grow and collect debris. In the case of a large stone or multiple stones, routes between a renal calyx and renal papillae can become inhibited, causing sever discomfort. Some stones or stone fragments can travel to the ureter and can cause considerable pain. Other stones can become too large to be passed through the ureter and must be removed by a surgical procedure such as percutaneous nephrolithotomy (PCNL). Kidney stones can form repeatedly in certain patients having predispositions to the formation of kidney stones such as hereditary factors, obesity, or diet. As such, routine treatments must be employed to help remove and to help mitigate stone formation especially in frequent kidney stone disease patients.

Several measures can be taken, in addition to dietary considerations such as increased hydration or lowering calcium intake, to reduce kidney stone formation. In one approach, oral medications can be administered such as to affect the chemical composition of fluid in the renal calyxes. For example, thiazide diuretics, citrate, allopurinol, of vitamin C supplements can be consumed to help prevent certain types of bioelements from forming kidney stones. In another example chemolysis can be achieved, such as by oral medications, antegrade nephrostomy, or retrograde ureteral catheters, to increase the pH of the urine and thus reduce the aggregation of certain calcium oxalate stones. A problem with these approaches is they can be ineffective to certain types of stones and can fail to adequately reduce aggregation or coagulation of minerals in the kidney. The present apparatus and techniques can help supply mechanical disruption in a human organ, for example the kidney, the common bile duct, the gall bladder, or other human organs where disruption would be medically beneficial and thus reduce stone formation of a variety of compositions.

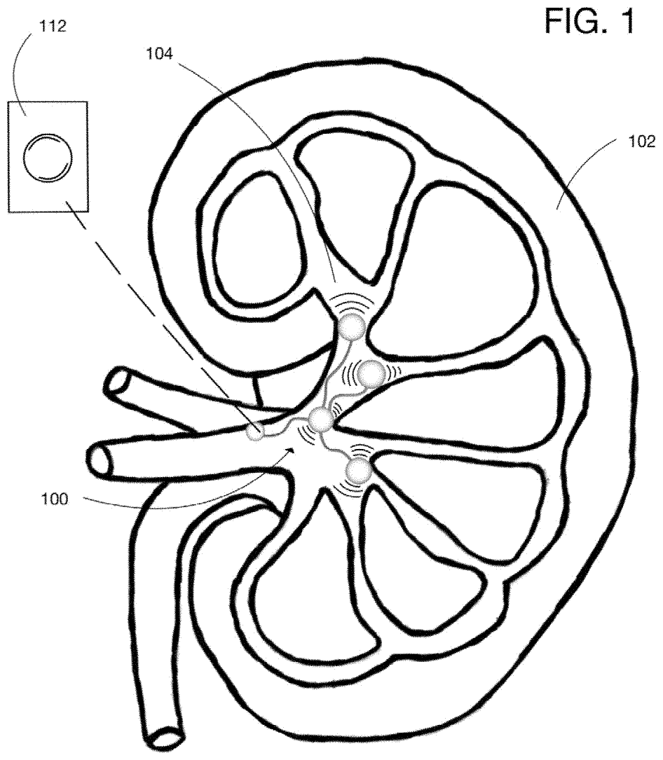

FIG. 1 shows a side view of an example of a stone disruptor in operation inside a human kidney. The stone disruptor can include or use one or more turbulence generators 100 operatively connected to an actuator 112. At least a portion of the stone disruptor, such as the turbulence generator 100 as depicted in FIG. 1, can be deployed into a renal pelvis 104 of a human kidney 102. In one example, the turbulence generator can be injected or implanted into the kidney 102 using a surgical procedure such as ureteroscopy. In one example, a ureteroscope or other minimally invasive surgical device can be provided such as to provide access to the kidney 102 through a body opening, cavity, or tract. A probe, which can be a needle, can create a passage such that a guide wire can be threaded from the surface of the skin to the surgical site. Later in the procedure, the initial insertion can be dilated such as to accommodate the surgical device. The surgical device can supply the portion of the stone disruptor, such as the turbulence generator 100, to the kidney 102. In one example of ureteroscopy, the ureteroscope can be a disposable. The ureteroscope can also be at least partially reusable and can be autoclaved or chemically sanitized. The ureteroscope can be sized and shaped to such as for urethral passage into the kidney. Also, the ureteroscope can be capable of transluminal passage into the kidney. The turbulence generator 100 can become implanted within the renal pelvis 104 of a human kidney 102 for a predetermined amount of time. In one example, the portion of the disruptor 100 can remain within the kidney 102 for about three months without maintenance and before removal. In another example, the portion of the disruptor 100 can remain within the kidney 102 for about one year without maintenance and before removal. In another example, the portion of the disruptor 100 can remain within the kidney 102 for about five years without maintenance and before removal. In yet another example, the turbulence generator 100 can remain within the kidney 102 indefinitely without the need for maintenance or removal. In some examples, the turbulence generator 100 can be routinely replaced, such as routinely surgically replaced. Removal of the portion of the disruptor 100 can involve a surgical procedure, similar to the implantation and deployment thereof. In another example, the at least part of the implanted portion of the disruptor, such as part of the turbulence generator 100 as depicted in FIG. 1, can be dissolvable, biodegradable, or fragmentable within the kidney and can be gradually passed through the urinary tract for partial, near complete, or complete removal of the portion of the disruptor 100 from the kidney 102.

The actuator 112 can be operatively connected to the turbulence generator 100. The actuator can function to manipulate the turbulence generator 100, such as to cause the generator 100 to oscillate or produce an acoustic wave in a medium within the renal pelvis. In one example, as depicted in FIG. 1, the actuator 112 can be located outside of the kidney 102 or outside of the human body and can include or use a wireless connection for communication with the turbulence generator 100. The wireless connection can be a BLUETOOTH connection, a Wi-Fi connection, a cellular connection, a radiofrequency connection, a magnetic force, or any other suitable wireless connection. In another example, the actuator 112 can be physically located at or near the turbulence generator 100 and thus, implantable into the kidney 102. Where the actuator 112 is located within the kidney 102, the actuator can include or use circuitry such as a digital processor, an analog timing mechanism, or other circuitry suitable for actuating the turbulence generator 100 at a predetermined time. In yet another example, the stone disrupter can include or use a turbulence generator 100 which produces oscillation constantly and can administer oscillation without being operatively connected to an actuator 112. In such examples, the stone disrupter can lack an actuator 112. Finally, in some examples, the stone disrupter can include or use a wired connection to operatively connect the actuator 112 with the turbulence generator 100.

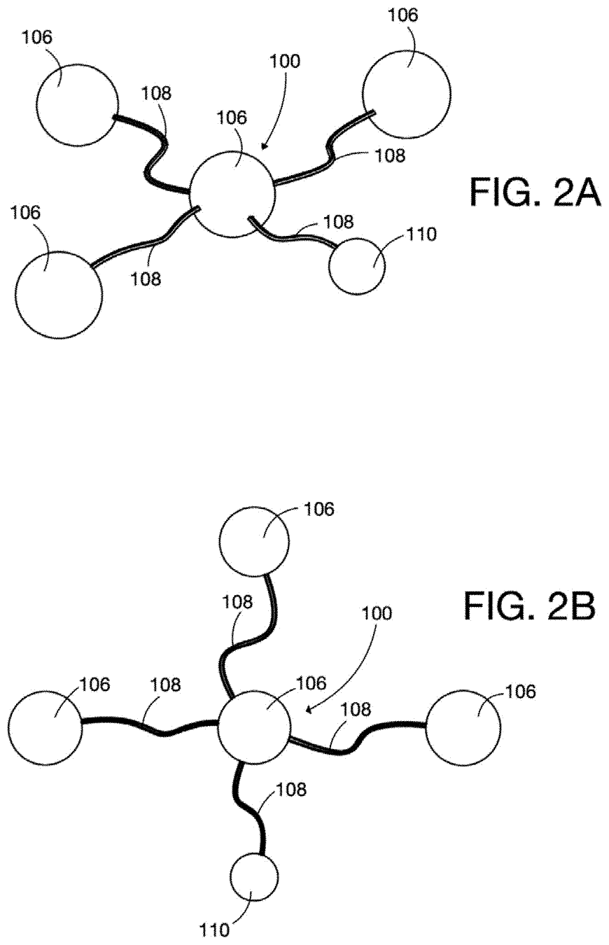

FIG. 2A and FIG. 2B show perspective and side views, respectively, of an example of a portion of the stone disruptor. A stone disruptor can include or use the turbulence generator 100. The turbulence generator 100 can include or use one or more elements 106 which can also be referred to as oscillation elements 106, and the oscillation elements 106 can function such as to produce an acoustic wave in a medium within the renal pelvis 104 (as depicted in FIG. 1). Turbulence produced by the oscillation elements 106 can reduce stagnation and circulate the kidney fluids out of the calyxs and into the main body of the kidney and then down the ureter. The oscillation elements 106 can be operatively connected by one or more tethers 108. The tethers 108 can transmit power, data, or both between oscillation elements 106. In one example, the turbulence generator 100 can further include or use a module 110, as depicted in FIG. 2A and FIG. 2B. The module 110 can similarly be operatively connected to one or more of the oscillation elements 106 by one or more tethers 108. The module 110 can house the circuitry, a power source, or both. In some examples, the module can additionally have components similar to those described herein with respect to the turbulence generator 100 and function to provide oscillation: herein, descriptions of the oscillation elements 106 can also include the module 110. In one example, the module 110 can be a hub and the oscillation elements 106 can extend, via the tethers 108, therefrom. In another example, as depicted in FIG. 2A and FIG. 2B, one of the oscillation elements 106 can be a hub and the module 100 or other oscillation elements 106 can extend therefrom via the tethers 108. Several oscillation elements 106 can extend with several degrees of freedom from one another, such as to extend from the hub into calyxes of the kidney. The tethers 108 can facilitate free or minimally restricted travel of the oscillation elements 106 around the renal pelvis 104 while still supplying power or data to each oscillation element 106.

The oscillation element 108 can include or use one or more mechanisms which can function to produce an acoustic wave in a medium within the renal pelvis. In one example, the oscillation element 108 can include or use a chassis, and electric motor, and an eccentric weight disposed on a drive shaft of the electric motor. The rotation of the eccentric weight by the turning of the drive shaft can cause the element 108 to oscillate. In another example, the oscillation element can include or use a chassis, a linear actuator, and a weight attached to a moving portion of the linear actuator. As the linear actuator operates, the weight attached to the moving portion of the linear actuator can cause the element 108 to oscillate along a linear path. The linear actuator can be an electric actuator, a piezoelectric actuator, a hydraulic actuator, a pneumatic actuator or can include or use a micro-electromechanical (MEMS) or microfluidics component. In other examples, the oscillation element 108 can be an acoustic transducer, electromagnetic coils, a piezoelectric vibrator, or other oscillating mechanism. In some examples, the oscillation element 108 can produce acoustic waves at frequencies between about 100 Hz and about 350 Hz. The oscillation element 108 can produce acoustic waves at frequencies between about 150 Hz and about 350 Hz. Alternatively or additionally, the oscillation element 108 can produce ultrasonic waves at frequencies greater than about 20,000 Hz. The oscillation element 108 can produce ultrasonic waves within a range of medically safe frequencies without causing any significant adverse clinical effect.

The oscillation element 108 can be operatively coupled to a source of power. In one example, the module 110 can house a battery and the battery can supply power to the oscillation elements 108. In another example, the source of power can be located outside the kidney 102 and can be tethered to the oscillation element by a connection. In yet another example, the source of power can be magnetic force wirelessly supplied by a controller. The controller can be located at or near the turbulence generator 100, or also can be located outside of the kidney 102. In a similar fashion, the source of power can be other wireless forces supplied by the controller such as an electromagnetic field, Lorentz forces, radio frequencies, or other frequencies outside of the visible spectrum.

The components of the turbulence generator 100, such as the oscillation element 108, the module 110, or the tethers 108 can be formed of materials suitable for contact with a human kidney. In one example, the components of the turbulence generator 100 can be formed of stainless steel, polytetrafluoroethylene (PTFE), silicon, superelastic shape-memory materials such as nitinol, chromium-cobalt based alloys, titanium and titanium based alloys, magnesium alloys, ceramic materials, polymeric materials, or one of several naturally biodegradable polymeric biomaterials such as proteins, polysaccharides, or native polyesters such as polyhydroxyalkanoates (PHA). The turbulence generator 100 can be formed of materials such that it can be struck with a secondary instrument and not fragment. Further, the turbulence generator 100 can be formed of materials such that it can be subject to energy from a lithotripter, laser, or other secondary instrument and not fragment. The turbulence generator 100 can also be formed with materials such that it can be struck with a secondary instrument or secondary instrument energy and still be completely retrieved safely from the renal pelvis 104. In some examples, one or more components of the turbulence generator 100 can be basket-shaped. The exterior surface of the turbulence generator 100 can be coated or laced with an anti-stone-forming material such as an antibiotic. In one example, the anti-stone-forming material can be paclitaxel.

Several approaches can be taken to ensure the turbulence generator 100 itself does not block the urinary tract or travel undesirably to the ureter. In some examples, the turbulence generator 100 can be at least partially attached or tethered to an interior wall of the renal pelvis 104 or to another surface in the urinary tract. For instance, the turbulence generator 100 can be at least partially attached or anchored to an interior bodily surface via a binding such as a suture, a clip, or a surgical glue. In another example, the turbulence generator 100 can be used with a stent, and the stent can be attached to an interior bodily surface such as a calyx of the kidney 102. In another example, the turbulence generator 100 can be used with a urethral stent. Alternatively, the turbulence generator 100 can be free-floating in the fluid of the renal pelvis 104 and unattached to any interior bodily surface. The apparatus can be sized and shaped such as to not become lodged within the calyxes of the renal pelvis 104.

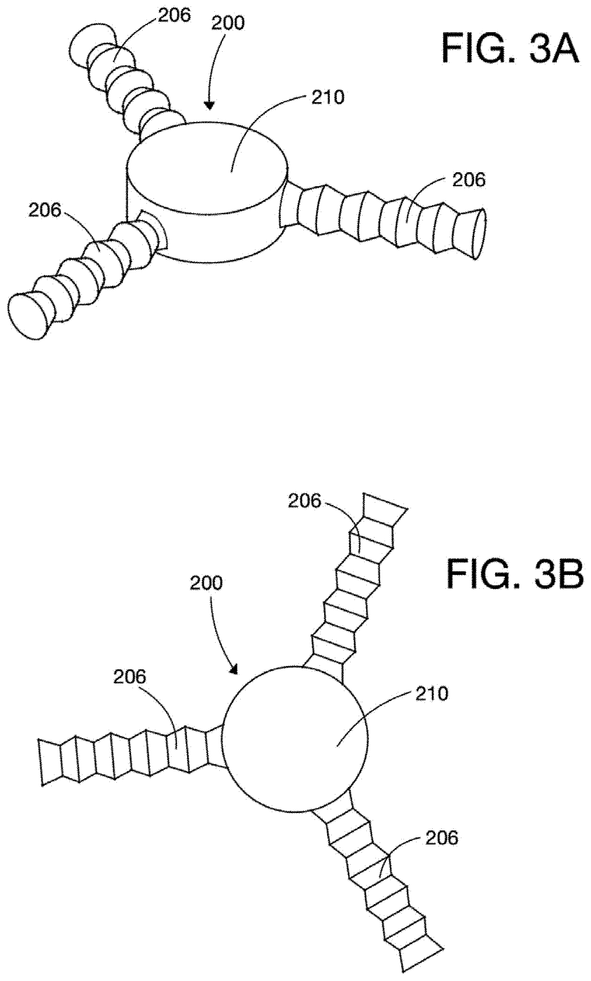

FIG. 3A and FIG. 3B show perspective and side views, respectively, of another example of a portion of the stone disruptor. A turbulence generator 200 can be similar in many respects to the turbulence generator 100. Here, the stone turbulence generator 200 can include or use a controller 210, which can be a hub, operatively connected to one or more oscillation elements 206. In some examples, the oscillation elements 206 can be directly operatively connect to the controller 210. The oscillation elements 206 can be sized and shaped as flexible appendages or fins that can extend from the controller 210 and into one or more calyxes of the renal pelvis 104. In several examples, the oscillation elements 206 can oscillate in a similar fashion to that of the oscillation elements 106. Alternatively or additionally, the oscillation elements 206 be connected to a drive shaft of an electric motor and can be rotated thereby with respect to the controller 210, thus generating turbulence of fluid within the renal pelvis 104.

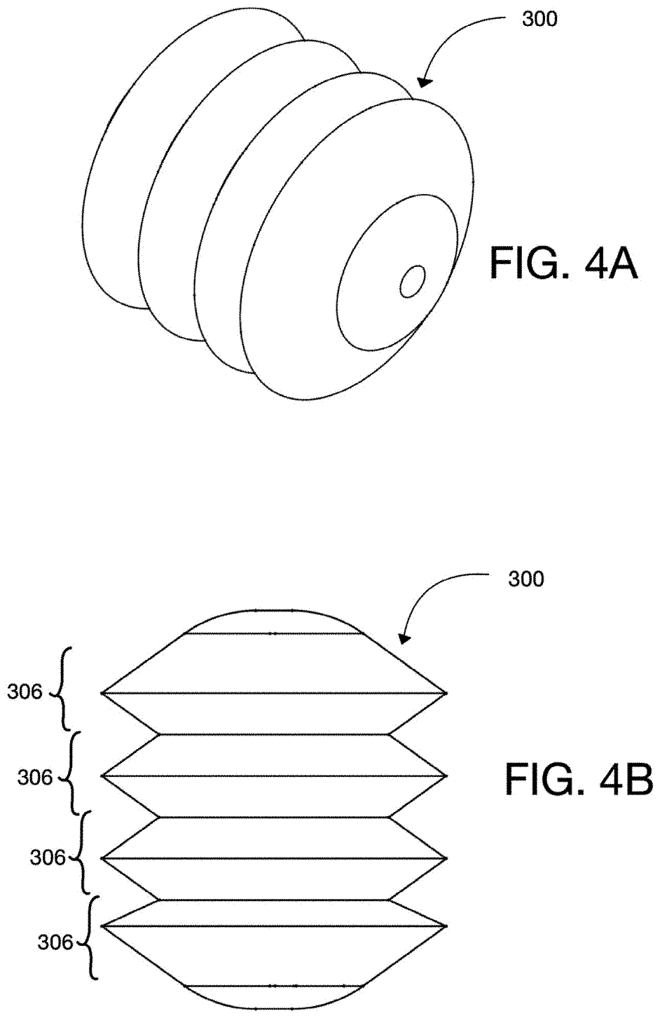

FIG. 4A and FIG. 4B show perspective and side views, respectively, of yet another example of a portion of the stone disruptor. The turbulence generator 300 can be similar in many respects to the turbulence generators 100 and 200. Here, the turbulence generator 300 can include or use one or more oscillation elements 306 arranged as a drum diaphragm. In several examples, the oscillation elements 306 can oscillate in a similar fashion to that of the oscillation elements 106 and 206. Alternatively or additionally, the oscillation elements 306 can collectively or individually expand and contract using electric, piezoelectric, hydraulic, or pneumatic energy. In so doing, the turbulence generator 300 can create pulsation and thus generate turbulence of fluid within the renal pelvis 104.

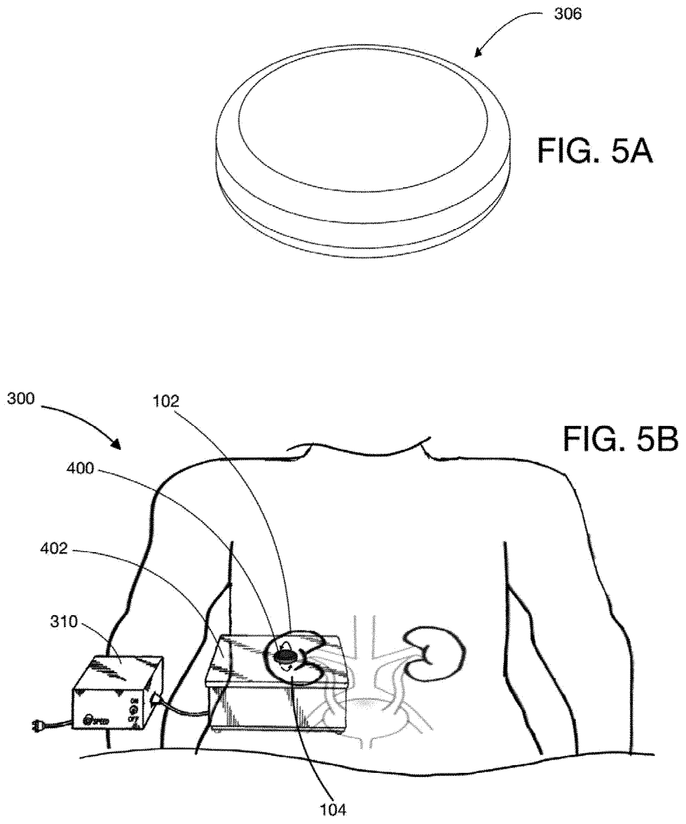

FIG. 5A and FIG. 5B show perspective and side operational views, respectively, of yet another example of a portion of the stone disruptor. In several examples, such as those depicted in FIG. 5A and FIG. 5B, the actuator can be located within an external manipulator 308, and the manipulator can include or use a housing and a plurality of magnetic drives located within the housing. The external manipulator 308 can include or use a controller 310 operatively coupled to the manipulator. The controller 310 can contain circuitry capable of sending signals to the external manipulator 308 such as to selectively actuate the magnetic drives in accordance with an actuation sequence. One or more oscillation elements 306 can be magnetically inclined to move in response to the magnetic drives being actuated such as to stir the medium or fluid within the renal pelvis 104. The external manipulator 308 can actuate the oscillation element 306 to spin, flip, rotate, or travel in the fluid medium or the renal pelvis 104. In one example, as depicted in FIG. 5A, the oscillation element 306 can be coin shaped. The oscillation element 306 can also be pill-shaped or can be formed in a non-symmetrical shape. In an example, the oscillation element 306 can include or use a source of power and electromagnetic coils capable of causing rapid movement in response to the magnetic drives being actuated. In other examples, the oscillation element 306 can be non-powered or passive and made of metallic or magnetic material. In several examples, the oscillation element 306 can be a magnetic stir element predisposed such as to move in response to the magnetic drives of the housing.

As depicted in FIG. 5B, the external manipulator 308 can be located outside of the human body and can function to wirelessly actuate the oscillation element 306 inside the renal pelvis 104. The external manipulator 308 can administer electromagnetic field or Lorentz forces such as to manipulate the oscillation element 306. In one example, the external manipulator 308 can be wearable by a human patient during actuation. For instance, the external manipulator 308 can be worn as part of a belt or strap and can be fastened or tied to the patient. In another example, the external manipulator 308 can be a portable, remote component such as a pod or wand. Also, the external manipulator 308 can be incorporated into a bed or a table. A patient can lie near the external manipulator 308 and receive actuation therefrom. The external manipulator 308 can either be remotely connected to the controller 310, or can incorporate the controller 310 within the housing of the external manipulator 308. The controller or the external manipulator can be coupled to a source of power, such as 120V AC power, or can be connected to batteries for supplying power for actuation. In a number of examples, the controller 310 or the external manipulator 308 can include or use controls or a user interface and circuitry connected thereto for operation of the actuation sequence. Alternatively or additionally, the external manipulator 308 can be magnetic and completely passive, requiring no electrical power, and still function such as to wirelessly, magnetically actuate or displace the oscillation element 306 inside the renal pelvis 104.

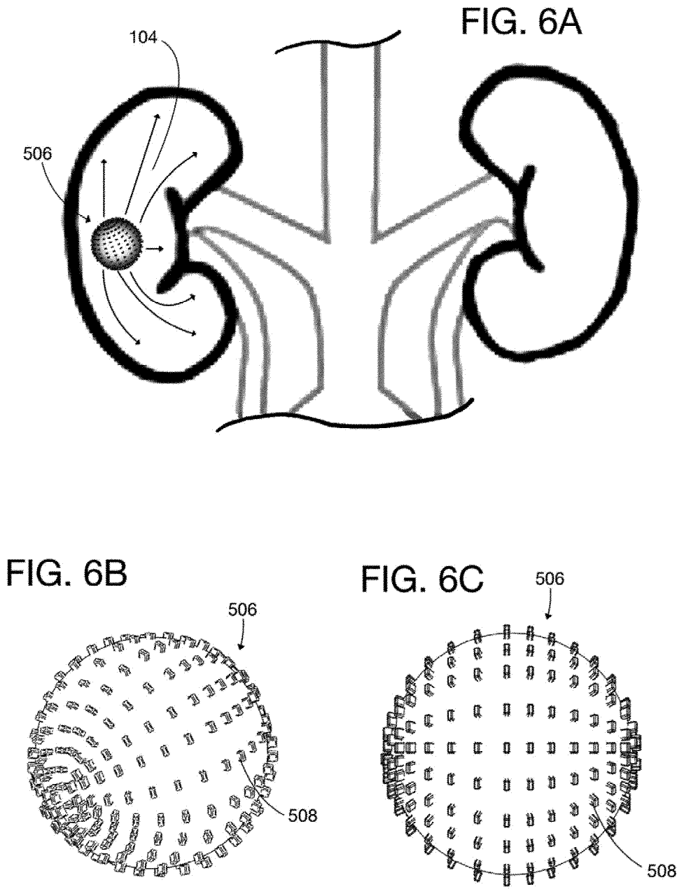

FIG. 6A, FIG. 6B, and FIG. 6C show a side operative view, a perspective view, and a side view of an example of a stone disruptor, respectively. The stone disruptor can be a turbulence generator 506. The turbulence generator 506 can be similar in several respects to the turbulence generators 106, 206, 306, and 406. Here, the turbulence generator 506 can be a passive element capable of producing turbulence without the need for oscillating, pulsing, vibrating, spinning, or other mechanical stimulation of the generator 506. The turbulence generator 506 can generate mechanical turbulence in the medium within the renal pelvis 104 and can circulate the medium out of calyxes into a main body of the renal pelvis 104 and down the ureter. Bodily motion from the patient, such as standing up, walking, or running, can cause the turbulence generator 506 to travel or bounce around the renal pelvis 104. As the patient moves, and because the element is suspending within the medium, at least one of gravitational or contact forces can propel the element to generate turbulence. The turbulence generator 506 can include or use protrusions 508 such as to further cause disruption of the fluid medium within the renal pelvis 104.

In operation and use, a turbulence generator can be provided or obtained such as for being deployed into a renal pelvis of a kidney to provide therapy or treatment for kidney stones. The turbulence generator can be deployed or administered into the renal pelvis by a medical procedure such as ureteroscopy. In some examples, the turbulence generator can be deployed into the renal pelvis using a surgical procedure. The turbulence generator can be activated such as to be suspended within a fluid medium of the renal pelvis for generation of mechanical turbulence. The fluid medium of the renal pelvis can be a naturally-occurring fluid within the renal pelvis before the medium is activated. The medium can be circulated out of calyxes into a main body of the renal pelvis and down a ureter. In some examples, Lorentze forces or electromagnetic forces can be administered from the turbulence generator and to the medium within the renal pelvis.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A medical apparatus for mitigating formation of kidney stones in a human patient, the apparatus comprising:
a turbulence generator deployable into a renal pelvis of a human kidney, the generator comprising an element configured to produce an acoustic wave in a medium within the renal pelvis; and
an actuator configured to manipulate the turbulence generator;
wherein one of the turbulence generator or the actuator is configured for coupling with a source of power.

2. The medical apparatus of claim 1, wherein the element comprises:
a chassis;
an electric motor; and
an eccentric weight disposed on a drive shaft of the electric motor;
wherein rotation of the eccentric weight by the drive shaft causes the element to oscillate.

3. The medical apparatus of claim 1, wherein the element comprises:
a chassis;
a linear actuator; and
a weight configured to be oscillated along a linear path by the linear actuator.

4. The medical apparatus of claim 1, wherein the element is an acoustic transducer.

5. The medical apparatus of claim 1, wherein the element comprises electromagnetic coils.

6. The medical apparatus of claim 1, wherein the element is a piezoelectric vibrator.

7. The medical apparatus of claim 1, wherein the actuator is located within an external manipulator, the manipulator comprising:
a housing;
a plurality of magnetic drives located within the housing; and
a controller that selectively actuates the magnetic drives in accordance with an actuation sequence;
wherein the element is configured to move in response to the magnetic drives being actuated to stir the medium within the renal pelvis.

8. The medical apparatus of claim 7, wherein the manipulator is configured to be located outside of a human body to actuate the element inside the renal pelvis of the human kidney.

9. The medical apparatus of claim 8, wherein the manipulator is wearable by a human patient during actuation.

10. The medical apparatus of claim 9, wherein the manipulator comprises a belt configured for fastening to a human patient.

11. The medical apparatus of claim 7, wherein the element is a magnetic stir element configured to move in response to the magnetic drives of the housing.

12. The medical apparatus of claim 8, wherein the element is coin-shaped.

13. The medical apparatus of claim 8, wherein the element is pill-shaped.

14. The medical apparatus of claim 8, wherein the element comprises a non-symmetrical shape.

15. The medical apparatus of claim 1, further comprising a binding configured to anchor the turbulence generator to a predetermined location within the kidney.

16. The medical apparatus of claim 1, wherein the element produces acoustic waves at frequencies between about 150 Hz and about 350 Hz.

17. The medical apparatus of claim 1, wherein the element produces ultrasonic waves at frequencies greater than about 20,000 Hz.

18. A medical apparatus for mitigating formation of kidney stones in a human patient, the apparatus comprising:
a turbulence generating element implantable into a renal pelvis of a human kidney and configured to passively generate turbulence in a medium within the renal pelvis;
wherein the element is suspended within the medium and at least one of gravitational or contact forces propel the element to generate the turbulence.

19. The medical apparatus of claim 18, further comprising a binding configured to anchor the turbulence generating element to a predetermined location within the kidney.

20. A method for treating a human patient, the method comprising:
deploying a turbulence generator into a renal pelvis of a kidney of the patient;
activating the turbulence generator;
generating mechanical turbulence in a medium within a renal pelvis, wherein the medium comprises a naturally-occurring fluid within the renal pelvis before the medium is activated; and
circulating the medium out of calyxes into a main body of the renal pelvis and down a ureter.

21. The method of claim 20, wherein generating mechanical turbulence comprises administering a Lorentz force or an electromagnetic force from the turbulence generator and to the medium within the renal pelvis.

22. The method of claim 21, wherein the turbulence generator is deployed into the renal pelvis using a ureteroscope.
